(19) United States
(12) Patent Application Publication
(10) Pub. No.: US 2025/0360340 A1
(43) Pub. Date: Nov. 27, 2025

(54) MONITORING ORGAN MORPHOLOGY FOR RADIATION TREATMENT DELIVERY USING ULTRASOUND

(71) Applicant: The Medical College of Wisconsin, Inc., Milwaukee, WI (US)

(72) Inventors: William Adrian Hall, Elm Grove, WI (US); Eric Paulson, Milwaukee, WI (US)

(21) Appl. No.: 19/288,971

(22) Filed: Aug. 1, 2025

Related U.S. Application Data
(63) Continuation of application No. PCT/US2024/014302, filed on Feb. 2, 2024.
(60) Provisional application No. 63/483,252, filed on Feb. 3, 2023.
(60) Provisional application No. 63/505,473, filed on Jun. 1, 2023.

(51) Int. Cl. A61N 5/10

(52) U.S. Cl. CPC A61N 5/1049; A61N 5/1037; A61N 2005/1058

(57) ABSTRACT

The morphology of an anatomical target is monitored using ultrasound to confirm when the anatomical target's morphology is substantially aligned, or otherwise in agreement, with a reference morphology indicated by a radiation treatment plan and determined from pre-treatment ultrasound data. Ultrasound data are acquired to measure and monitor the morphology of an anatomical target, such as the bladder, rectum, stomach, or the like. The ultrasound data are compared to reference data. When the morphology of the anatomical target is substantially aligned, or otherwise in agreement, with the anatomical morphology indicated by the reference data and the radiation treatment plan, then the patient can be provided with the planned radiation treatment for that day.

### CROSS-REFERENCE TO RELATED APPLICATIONS

This application is a continuation of International Application No. PCT/US2024/014302, filed Feb. 2, 2024, which claims the benefit of U.S. Provisional Patent Application Ser. No. 63/483,252, filed on Feb. 3, 2023, and entitled “Monitoring Organ Morphology for Radiation Treatment Delivery Using Ultrasound,” and U.S. Provisional Patent Application Ser. No. 63/505,473, filed on Jun. 1, 2023, and entitled “Monitoring Organ Morphology for Radiation Treatment Delivery Using Ultrasound,” both of which are herein incorporated by reference in their entirety.

### BACKGROUND

Accurate delivery of radiation therapy to patients requires repeatable patient positioning and precise knowledge of the morphology of internal organs surrounding the planned target volume. Because of this, radiation therapy for patients with pelvic malignancies such as prostate cancer, anal cancer, rectal cancer, endometrial cancer, pelvic sarcoma, or cervical cancer is preferably delivered when the patient's bladder is comfortably full and the rectum is empty. Similarly, upper abdominal tumors (e.g., pancreatic cancer, esophageal cancer, or primary/secondary liver tumors) require reproducible gastric emptying. There may be some instances, however, where a different size and/or filling of the regional organ (e.g., bladder, rectum, stomach) can provide more optimal alignment of the planned target volume with reduced exposure to surrounding organs-at-risk. Understanding and optimally filling/emptying the regional organs-at-risk is an important, yet challenging, part of patient preparation for radiation treatment.

Maintaining a consistent size of regional organs-at-risk (e.g., bladder, rectum, stomach) filling during both treatment planning and delivery is challenging for patients. For example, when considering the bladder, some techniques for maintaining a consistent bladder filling include administering a catheter to the patient, where the catheter drains the bladder only when it is filled beyond a prescribed volume. However, this approach places that patient at undesirable risk for potential infection or trauma associated with catheter introduction.

There remains a need for more accurate, repeatable, and less invasive techniques for monitoring the morphology of anatomical targets that affect the delivery of external beam radiation treatments.

### SUMMARY OF THE DISCLOSURE

The present disclosure addresses the aforementioned drawbacks by providing a method for analyzing ultrasound data to monitor alignment of an anatomical target morphology with a radiation treatment plan. The method includes acquiring ultrasound data from a region-of-interest in a patient containing an anatomical target, where the ultrasound data are acquired using a plurality of ultrasound transducer arranged about the region-of-interest. Reference ultrasound data are accessed with a computer system, which may include a user device. The reference ultrasound data were acquired from the patient while the anatomical target was maintained at a specified reference morphology indicated by a radiation treatment plan. The ultrasound data are compared with the reference ultrasound data, generating feedback data that indicate a conformance of the anatomical target morphology to the reference morphology. When the anatomical target morphology is optimally aligned with the reference morphology, a notification is generated and delivered to the patient via a user device, where the notification indicates that the anatomical target morphology is optimally aligned with the reference morphology indicated by the radiation treatment plan.

It is another aspect of the present disclosure to provide a user device that includes a display, a memory, and a processor in communication with the display and the memory. The processor is configured to: receive ultrasound data acquired from a region-of-interest in a patient containing an anatomical target, wherein the ultrasound data were acquired using a plurality of ultrasound transducer arranged about the region-of-interest; access from the memory, reference ultrasound data acquired from the patient while the anatomical target was maintained at a specified reference morphology indicated by a radiation treatment plan; compare the ultrasound data with the reference ultrasound data, generating feedback data that indicate a conformance of the anatomical target morphology to the reference morphology; generate a user interface displayed to a user by the display; and present the feedback data in the user interface displayed by the display.

It is yet another aspect of the present disclosure to provide a method for monitoring agreement of an anatomical target morphology with a radiation treatment plan. The method includes accessing ultrasound data with a computer system, where the ultrasound data have been acquired from a region-of-interest in a patient containing an anatomical target. Reference ultrasound data are also accessed with the computer system, where the reference ultrasound data have been acquired from the patient while the anatomical target was maintained at a specified reference morphology indicated by a radiation treatment plan. Feedback data are generated with the computer system by comparing the ultrasound data with the reference ultrasound data. The feedback data indicate an agreement of the anatomical target morphology to the reference morphology. The feedback data are then output to a user via the computer system.

### BRIEF DESCRIPTION OF THE DRAWINGS

FIG. 1 is a block diagram of an example system for monitoring anatomical target morphology using a wearable ultrasound device.

FIG. 2 is a flowchart illustrating the steps of an example method for monitoring the morphology of an anatomical target in a patient who is to receive radiation treatment.

FIG. 3 is an example of ultrasound data that can be acquired with a wearable ultrasound device including five different ultrasound transducers.

FIG. 4 is a block diagram of an example anatomical target morphology monitoring and alignment system in accordance with some embodiments described in the present disclosure.

FIG. 5 is a block diagram of example components that can implement the system of FIG. 4.

### DETAILED DESCRIPTION

Described here are systems and methods for radiation treatment planning and delivery based on monitoring anatomical morphology using ultrasound. In general, on a day that a patient is to be administered a radiation treatment dose, they are provided with a device including multiple ultrasound transducers that can be worn around an region-of-interest (ROI). Ultrasound data are acquired with the device to measure and monitor the morphology of an anatomical target in the ROI, such as the bladder, rectum, stomach, or the like. The ultrasound data are compared to reference data acquired from the patient before the treatment day (e.g., reference ultrasound data that have been benchmarked against acceptable anatomy at the time of simulation), where these reference data were also used, at least in part, to generate a radiation treatment plan for the patient. When the morphology of the anatomical target is substantially aligned (e.g., aligned within tolerances allowed by the radiation treatment plan) or otherwise in agreement with the anatomical morphology indicated by the reference data and the radiation treatment plan, then the patient can be provided with the planned radiation treatment for that day. In this way, a higher level of accuracy in the delivery of radiation treatment can be provided. Additionally or alternatively, the disclosed systems and methods can improve clinical throughput, patient satisfaction, and reduce the amount of scans needed to confirm normal organ position for radiation treatment.

As one non-limiting example, the anatomical target may be a bladder. In these instances, the ultrasound data acquired on the treatment day can be used to monitor the morphology of the bladder relative to the morphology of the bladder on the pre-treatment day when the radiation treatment plan was developed. For example, the patient may be instructed on the pre-treatment day to drink a volume of water or other liquid until their bladder is filled to a volume that is optimized for the planned radiation delivery. An optimized volume may include a volume that results in a bladder morphology that optimally aligns a planned treatment volume (PTV) (e.g., a prostate, or other anatomical location being treated) with the radiation delivery beam. Optimally aligning the PTV can include aligning the PTV while minimizing exposure of organs-at-risk (OARs).

Thus, in general, the systems and methods described in the present disclosure seek to monitor the morphology of an anatomical target, such that a nearby PTV can be optimally aligned with a radiation beam for treatment; that is, such that the PTV in the radiation treatment plan is in alignment with a radiation beam path while minimizing exposure of OARs proximate to the anatomical target. By closely monitoring the anatomical morphology, a repeatable patient setup can be achieved with higher accuracy than simply placing the patient in a similar position relative to the radiation beam. In particular, by monitoring the internal morphology of anatomy that can affect the location of the PTV, the repeatability of the patient setup can be further improved. Monitoring the morphology of an anatomical target (or more than one anatomical target) includes monitoring the volume, the position of the anatomical target, and the effect that the morphology of the anatomical target has on the surrounding anatomy, including the PTV.

As an example, the ultrasound device can acquire data indicative of the morphology of one or more internal organs. The ultrasound data, or a report generated therefrom, can be displayed to the patient via a user interface on a user device. As one non-limiting example, the ultrasound data and/or report can be presented to the user via a user device that may include a computer, a laptop, a smartphone, a tablet, or the like. In some implementations, the user interface can be generated as part of an application (app) running on the user device. The ultrasound data and/or report can provide the patient with feedback on the morphology of the anatomical target, including the size and shape of the organ. This data will then help the patient prepare for radiation treatment by giving them feedback on whether the morphology of the anatomical target is correctly, or otherwise optimally, aligned as required dosimetrically by the radiation treatment plan.

In some implementations, the user device can generate feedback data that is presented to the patient via the user device. For example, the user device can process the ultrasound data to generate feedback data that indicate the rate at which the morphology of the anatomical target is changing (e.g., via bladder filling, rectal filling, stomach filling, inhaling/exhaling, or the like). The feedback data can be further processed by the user device to generate a report that is presented to the patient via the user device, where the report indicates a prediction of the optimal time that the patient should be brought back for treatment (i.e., the time at which the morphology of the anatomical target will result in optimal conformance with the radiation treatment plan).

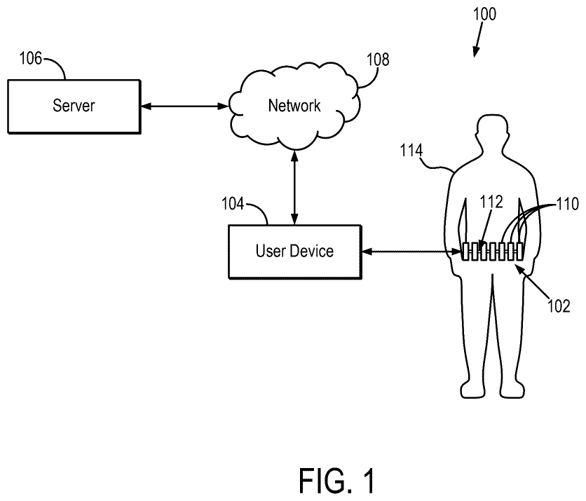

Referring now to FIG. 1, an example system 100 for measuring the morphology of an anatomical target is illustrated. In the illustrated embodiment, the system 100 includes an ultrasound device 102, a user device 104, a server 106, and a network 108.

In the illustrated embodiment, the ultrasound device 102 generally includes a wearable ultrasound device containing a plurality of ultrasound transducers 110. The ultrasound transducers 110 may be coupled together. For example, the ultrasound transducers 110 can be coupled together by a band 112, or the like. The band 112 can be fit around a region-of-interest on the patient 114 to hold the ultrasound transducers 110 in position about an anatomical target to be measured by ultrasound. Advantageously, by having a plurality of ultrasound transducer 110 that are arranged about the anatomical target in different positions and orientations, a more complete measurement of the morphology of the anatomical target can be acquired.

In other embodiments, the ultrasound device 102 can include a single ultrasound transducer that is wearable by the patient. Alternatively, the ultrasound device 102 can include one or more ultrasound transducers that are not configured to be wearable by the patient. For example, the ultrasound device 102 can include a handheld ultrasound transducer that can be operated by the patient or another user (e.g., a healthcare provider). In these instances, the ultrasound device 102 may be directly in communication with the user device 104 and/or server 106, or may be in communication with an ultrasound system that first acquires ultrasound data before passing the ultrasound data, or other data generated by processing the ultrasound data with the ultrasound system, to the user device 104 and/or server 106.

The ultrasound device 102 is in communication with a user device 104, which may be a computer, a laptop, a smartphone, a tablet, a smart watch, or other such device. The ultrasound device 102 can be in communication with the user device 104 via a wired connection, a wireless connection, or combinations thereof. Ultrasound data acquired by the ultrasound transducers 110 are transmitted to the user device 104, which then processes the ultrasound data to monitor or otherwise measure the morphology of the anatomical target.

The user device 104 can generate a user interface (e.g., a graphical user interface (UI)) to enable the patient 114 to control the operation of the ultrasound device 102, to monitor the ultrasound data received from the ultrasound device 102, to view reports or other feedback data generated from the ultrasound data, to provide patient feedback for the clinical team (e.g., feedback on patient comfort based on current filling of the anatomical target), and so on. For example, the user device 104 can run an app that provides a user interface for the patient 114 to control the ultrasound device 102 and/or to view data received therefrom. In some instances, the user device 104 can generate feedback data by processing the ultrasound data received from the ultrasound device 102, and can generate an alert, alarm, or other notification to the patient based on the currently measured morphology of the anatomical target. As an example, the user device 104 can generate a notification when the morphology of the anatomical target aligns, or substantially aligns, or is otherwise in agreement with a reference morphology (e.g., the morphology of the anatomical target indicated in a previously generated radiation treatment plan). Additionally or alternatively, the user device 104 can generate a notification indicating a rate of change in the morphology of the anatomical target, such as a size or shape of the anatomical target. This notification may also include a timer predicting when the morphology of the anatomical target is likely to align with the reference, which can inform the patient 114 about how soon they will be ready to receive radiation treatment. For instance, the timer can count down a predicted time to when the anatomical target morphology will optimally align with the reference morphology.

Additionally or alternatively, the user device 104 can run an app that provides a user interface (e.g., a graphical user interface) for the patient 114 to generate patient feedback data that may be used to provide updates to a clinical team, to adjust targets for the morphology of the anatomical target (e.g., by relaxing the amount of alignment required with the reference morphology based on patient comfort), or the like. For example, as the patient 114 receives feedback data on the user device 104 about the morphology of the anatomical target, the patient 114 can generate their own patient feedback data via the user device 104 to indicate their level of comfort based on the filling of the anatomical target. In this way, the patient is able to alert the clinical team about whether the proposed morphology for the anatomical target is too uncomfortable for the patient 114 to attain. This two-way feedback between the patient 114 and the clinical team allows for improved alignment with treatment planning in addition to improved patient comfort.

In some embodiments, the user device 104 may include a long-range transceiver to communicate with the server 106 and/or a short-range transceiver to communicate with other external devices via, for example, a short-range communication protocol such as Bluetooth® or Wi-Fi®. In some embodiments, the user device 104 bridges the communication between the ultrasound device 102 and the server 106. For example, the ultrasound device 102 may transmit data to the user device 104, and the user device 104 may forward the data from the ultrasound device 102 to the server 106 over the network 108.

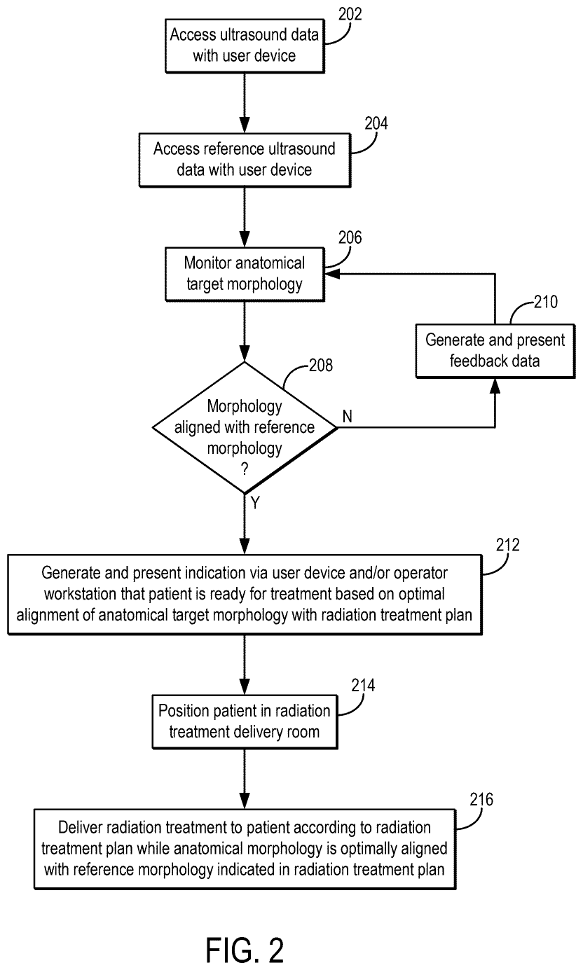

To perform its various functions, the user device 104 may include an electronic control assembly having an electronic processor, a memory, and a transceiver. The electronic processor may be configured to receive communications transmitted by the ultrasound device 102, process the data, store the data in the memory, generate notifications to the patient 114, and the like. The electronic processor and memory may collectively form a device electronic controller that is configured to perform certain methods described herein (e.g., FIG. 2).

The server 106 includes a server electronic control assembly having a server electronic processor, a server memory, and a transceiver. The transceiver allows the server 106 to communicate with the user device 104. The server electronic processor receives ultrasound data and/or other data from the ultrasound device 102 and/or user device 104, and stores the received data in the server memory. The server 106 may also maintain a database (e.g., on the server memory) for containing ultrasound data, reference ultrasound data, radiation treatment plan data, and so on. For instance, the server 106 can store reference ultrasound data that can be accessed by the user device 104 for comparing the ultrasound data received from the ultrasound device 102 to monitor the conformance of the morphology of the anatomical target to the radiation treatment plan.

Although illustrated as a single device, the server 106 may be a distributed device in which the server electronic processor and server memory are distributed among two or more units that are communicatively coupled (e.g., via the network 108).

The network 108 may be a long-range wireless network such as the Internet, a local area network (LAN), a wide area network (WAN), or a combination thereof. In other embodiments, the network 108 may be a short-range wireless communication network, and in yet other embodiments, the network 108 may be a wired network using, for example, USB cables. Additionally or alternatively, the network 108 may include a combination of long-range, short-range, and/or wired connections. In some embodiments, the network 108 may include both wired and wireless devices and connections.

Referring now to FIG. 2, a flowchart is illustrated as setting forth the steps of an example method for monitoring the morphology of an anatomical target in a patient who is to receive radiation treatment. An ultrasound device is used to acquire ultrasound data from the anatomical target, which are then analyzed to determine and monitor the morphology of one or more anatomical targets of interest. An alert, notification, or other report is presented to the patient to indicate when the morphology of the anatomical target is substantially aligned (or otherwise in conformance or agreement with) a reference morphology indicated in a radiation treatment plan.

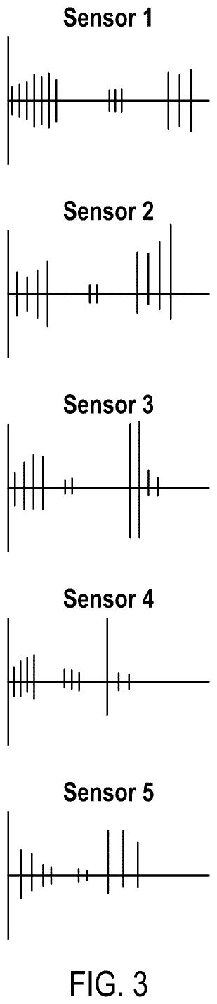

The method includes accessing ultrasound data with a user device, as indicated at step 202. Accessing the ultrasound data may include retrieving such data from a memory or other suitable data storage device or medium. Additionally or alternatively, accessing the ultrasound data may include acquiring such data with an ultrasound device and transferring or otherwise communicating the data to the user device. An example of ultrasound data acquired with an ultrasound device is illustrated in FIG. 3.

In general, the ultrasound data are acquired on the day that the patient is scheduled to receive radiation treatment. For instance, the ultrasound data are acquired while the patient is waiting to receive radiation treatment, but before they are positioned in the radiation treatment room. That is, in some implementations, the ultrasound data can be acquired while the patient is at the treatment facility, but before they are positioned in the treatment delivery room. Additionally or alternatively, the ultrasound data can be acquired while the patient is at home. In these instances, the patient can practice filling to achieve the optimal anatomical morphology.

The ultrasound data are acquired while the patient is waiting for radiation treatment or at home practicing filling, and may be acquired continually, or intermittently according to a timed schedule (e.g., an intermittent schedule). In some embodiments, the ultrasound data can be acquired on-demand in response to a user input for the ultrasound device to acquire data.

The method also includes accessing reference ultrasound data with the user device, as indicated at step 204. Accessing the reference ultrasound data may include retrieving such data from a memory or other suitable data storage device or medium. In some instances, the reference ultrasound data can be stored locally on the user device. In other instances, the reference ultrasound data can be stored remotely, such as on a server that is accessible by the user device.

In general, the reference ultrasound data are acquired during a pre-treatment planning session. The reference ultrasound data can be acquired with other imaging data, such as computed tomography (CT) data. When the anatomical target to be monitored is the patient's bladder, the reference ultrasound data are preferably acquired after the patient has been instructed to drink a volume of liquid (e.g., water) to provide optimal filling of their bladder, to empty their rectum, to wait a duration of time without eating to empty their stomach, or to consume food (which may include drinking a volume of liquid such as water) to provide optimal filling of their stomach. As described above, an optimal filling is one in which the morphology of the patient's bladder results in an optimal treatment trajectory to the PTV with minimized risk to surrounding OARs. In some examples, the reference morphology can be confirmed in the reference ultrasound data based on additional medical imaging of the patient. For instance, the reference morphology can be confirmed by computed tomography (CT) imaging of the patient, magnetic resonance imaging (MRI) of the patient, or the like.

The ultrasound data are compared with the reference ultrasound data to monitor the morphology of the anatomical target relative to the reference morphology indicated in the radiation treatment plan, as indicated at step 206. The ultrasound data are compared in real-time as they are being acquired or otherwise accessed. A check is made to determine whether the morphology of the anatomical target substantially aligns or otherwise conforms to or is in agreement with the reference morphology indicated by the reference ultrasound data, as determined at decision block 208. When the morphology of the anatomical target is not aligned with, conformed to, or in agreement with the reference morphology, feedback can be presented to the patient via the user device. For instance, a report can be displayed, or an alert or notification can be generated, as indicated at step 210. Additionally or alternatively, the report, alert, or notification can be generated and presented to a clinician, technician, or other healthcare provider. For instance, the feedback data or corresponding report, alert, or notification can be presented to the radiation therapy system operator, such that the operator can initiate radiation treatment delivery when the optimal anatomical morphology is achieved. In still other implementations, the feedback data may be transmitted to the radiation therapy system, which may then process the feedback data to control operation of the radiation beam according to a prescribed radiation treatment plan. As noted above, the feedback data may include an indication whether the morphology of the anatomical target is aligned with, conformed to, or in agreement with the reference morphology, a rate of change of the anatomical target's morphology, a prediction of when the anatomical target's morphology will align with, conform to, or otherwise be in agreement with the reference morphology, or combinations thereof. In some embodiments, the report, alert, or notification can indicate that the patient needs to drink more liquid, to urinate, to defecate, or so on, in order to achieve the optimal morphology of the anatomical target.

In still other examples, as described above, the patient may also provide patient feedback via the user device. In these instances, the patient feedback may be stored as patient feedback data that can be used to generate an alert or other notification to the clinical team, to adjust the target morphology of the anatomical target, or the like. For instance, the patient feedback can be used to adjust the alignment target between the morphology of the anatomical target and the reference morphology based on the patient feedback data. The patient feedback data may also include an indication of the patient's comfort level based on the current filling of the anatomical target (e.g., the current morphology of the anatomical target). In this way, the patient feedback data may be used to revise the target morphology for the anatomical target to account for patient comfort in addition to the degree of alignment with the reference morphology. In some instances, it may be acceptable to relax the degree to which the morphology of the anatomical target aligns with the reference morphology. For example, the morphology of the anatomical target may be out of alignment with the reference morphology within a certain tolerance. The patient feedback data may be used to identify whether the target morphology can be relaxed within these acceptable tolerances. Alternatively, the patient feedback data can be processed to generate an alert and/or notification to the clinical team. For instance, when the patient cannot comfortably attain the target morphology, or a revised morphology within the acceptable tolerances, then an alert and/or notification can be sent to the clinical team indicating that the target morphology and/or treatment plan may need to be adjusted to allow for patient comfort. Additionally or alternatively, a notification can be sent to the clinical team that the patient is unable to attain, or maintain, the target morphology of the anatomical target based on their comfort level. In these instances, the notification may alert the clinical team that the patient needs to be treated now (whether the anatomical target has achieved complete alignment with the reference morphology or not), or that treatment may need to be postponed to another day.

When the morphology of the anatomical target aligns with, conforms to, or is otherwise in agreement with the reference morphology, that feedback is presented to the patient via the user device, as indicated at step 212. For instance, a report can be displayed, or an alert or notification can be generated, which indicates that the morphology of the anatomical target has aligned with, conformed to, or otherwise has been made in agreement with the reference morphology. The notification presented to the patient may also indicate that the user should drink more liquid, fast, empty their bladder, and/or empty their rectum to help conform the morphology of the anatomical target to the reference morphology. Additionally or alternatively, as noted above, the feedback data may also be presented to the clinician, healthcare technician, or other healthcare provider via the treatment console or operator workstation. For instance, the feedback data can be sent to the treatment machine notifying the operator or other healthcare provider that the patient is ready for their daily treatment. In still other implementations, the feedback data may be transmitted to the radiation treatment system where they can be processed by a processor or controller of the radiation treatment system in order to control the operation of the radiation treatment system (e.g., by turning the radiation beam on/off in accordance with a prescribed radiation treatment plan and when the morphology of the anatomical target aligns with, conforms to, or otherwise is in agreement with the reference morphology).

The patient can then be positioned in the radiation treatment delivery room, as indicated at step 214, and radiation treatment can be delivered according to the previously generated radiation treatment plan, as indicated at step 216.

In this way, the disclosed method provides for timing reference/simulation data (e.g., CT image data, MRI data, ultrasound data) for optimal anatomical positioning of the patient; for timing radiation for maximally aligning treat-time CT anatomical morphology to reference morphology; or combinations thereof. Advantageously, these methods allow for optimal radiotherapy delivery by timing reference CT and/or ultrasound to optimal anatomical positioning or morphology; by minimizing ionizing radiation from unnecessary CTs; and by increasing clinic efficiency, reducing costs/labor, and easing logistics.

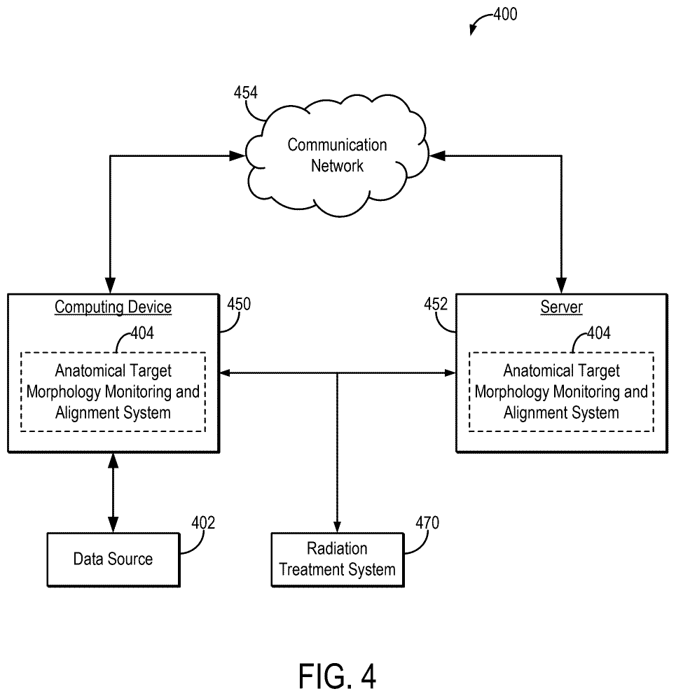

Referring now to FIG. 4, an example of a system 400 for processing ultrasound data to determine and monitor morphology of an anatomical target in accordance with some embodiments of the systems and methods described in the present disclosure is shown. As shown in FIG. 4, a computing device 450 can receive one or more types of data (e.g., ultrasound data, reference ultrasound data, pre-treatment CT or MRI image data, radiation treatment plan data, patient feedback data) from data source 402. In some embodiments, computing device 450 can execute at least a portion of an anatomical target morphology monitoring and alignment system 404 to monitor the alignment of an anatomical target's morphology relative to a reference morphology indicated in a radiation treatment plan from data received from the data source 402.

Additionally or alternatively, in some embodiments, the computing device 450 can communicate information about data received from the data source 402 to a server 452 over a communication network 454, which can execute at least a portion of the anatomical target morphology monitoring and alignment system 404. In such embodiments, the server 452 can return information to the computing device 450 (and/or any other suitable computing device) indicative of an output of the anatomical target morphology monitoring and alignment system 404.

In some embodiments, computing device 450 and/or server 452 can be any suitable computing device or combination of devices, such as a desktop computer, a laptop computer, a smartphone, a tablet computer, a wearable computer, a server computer, a virtual machine being executed by a physical computing device, and so on. The computing device 450 and/or server 452 can also reconstruct images from the data.

The computing device 450 and/or server 452 can also be in communication with a radiation treatment system 470, such as a linear accelerator (linac), proton treatment system, or other external beam treatment system. In these instances, the computing device 450 and/or server 452 can communicate information about the alignment of the anatomical target's morphology relative to the reference morphology indicated in the radiation treatment plan, and can assist in controlling the operation of the radiation treatment system 470 to deliver radiation treatment to the patient.

In some embodiments, data source 402 can be any suitable source of data (e.g., measurement data, images reconstructed from measurement data, processed image data), such as an ultrasound device, another computing device (e.g., a server storing measurement data, images reconstructed from measurement data, processed image data), and so on. In some embodiments, data source 402 can be local to computing device 450. For example, data source 402 can be incorporated with computing device 450 (e.g., computing device 450 can be configured as part of a device for measuring, recording, estimating, acquiring, or otherwise collecting or storing data). As another example, data source 402 can be connected to computing device 450 by a cable, a direct wireless link, and so on. Additionally or alternatively, in some embodiments, data source 402 can be located locally and/or remotely from computing device 450, and can communicate data to computing device 450 (and/or server 452) via a communication network (e.g., communication network 454).

In some embodiments, communication network 454 can be any suitable communication network or combination of communication networks. For example, communication network 454 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 454 can be a local area network, a wide area network, a public network (e.g., the Internet), a private or semi-private network (e.g., a corporate or university intranet), any other suitable type of network, or any suitable combination of networks. Communications links shown in FIG. 4 can each be any suitable communications link or combination of communications links, such as wired links, fiber optic links, Wi-Fi links, Bluetooth links, cellular links, and so on.

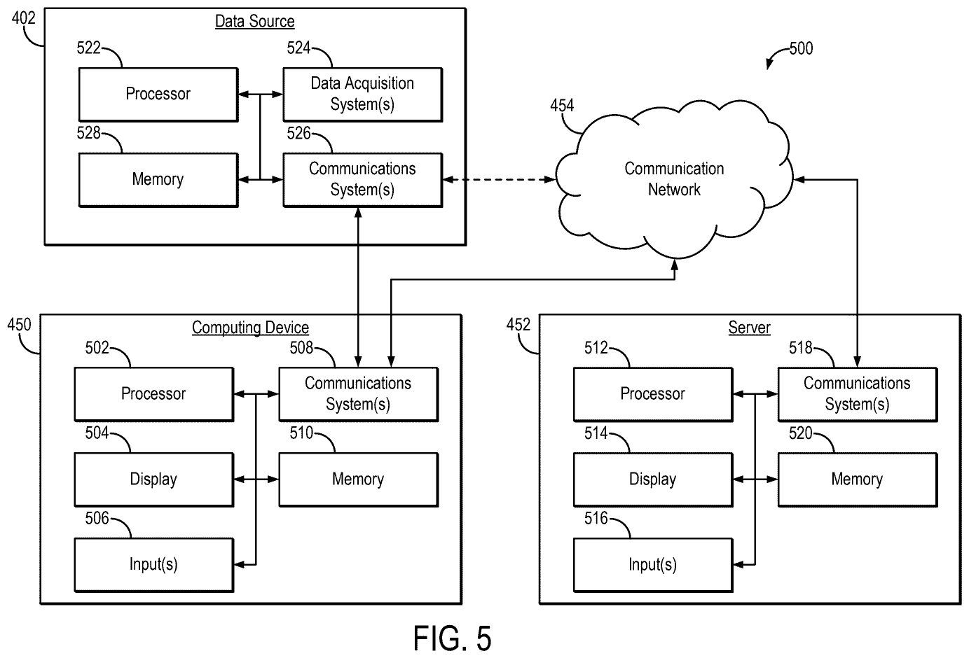

Referring now to FIG. 5, an example of hardware 500 that can be used to implement data source 402, computing device 450, and server 452 in accordance with some embodiments of the systems and methods described in the present disclosure is shown.

As shown in FIG. 5, in some embodiments, computing device 450 can include a processor 502, a display 504, one or more inputs 506, one or more communication systems 508, and/or memory 510. In some embodiments, processor 502 can be any suitable hardware processor or combination of processors, such as a central processing unit (CPU), a graphics processing unit (GPU), and so on. In some embodiments, display 504 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 506 can include any suitable input devices and/or sensors that can be used to receive user input, such as a keyboard, a mouse, a touchscreen, a microphone, and so on.

In some embodiments, communications systems 508 can include any suitable hardware, firmware, and/or software for communicating information over communication network 454 and/or any other suitable communication networks. For example, communications systems 508 can include one or more transceivers, one or more communication chips and/or chip sets, and so on. In a more particular example, communications systems 508 can include hardware, firmware, and/or software that can be used to establish a Wi-Fi connection, a Bluetooth connection, a cellular connection, an Ethernet connection, and so on.

In some embodiments, memory 510 can include any suitable storage device or devices that can be used to store instructions, values, data, or the like, that can be used, for example, by processor 502 to present content using display 504, to communicate with server 452 via communications system(s) 508, and so on. Memory 510 can include any suitable volatile memory, non-volatile memory, storage, or any suitable combination thereof. For example, memory 510 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 510 can have encoded thereon, or otherwise stored therein, a computer program for controlling operation of computing device 450. In such embodiments, processor 502 can execute at least a portion of the computer program to present content (e.g., images, user interfaces, graphics, tables), receive content from server 452, transmit information to server 452, and so on. For example, the processor 502 and the memory 510 can be configured to perform the methods described herein (e.g., the method of FIG. 2).

In some embodiments, server 452 can include a processor 512, a display 514, one or more inputs 516, one or more communications systems 518, and/or memory 520. In some embodiments, processor 512 can be any suitable hardware processor or combination of processors, such as a CPU, a GPU, and so on. In some embodiments, display 514 can include any suitable display devices, such as an LCD screen, LED display, OLED display, electrophoretic display, a computer monitor, a touchscreen, a television, and so on. In some embodiments, inputs 516 can include any suitable input devices and/or sensors that can be used to receive user input, such as a keyboard, a mouse, a touchscreen, a microphone, and so on.

In some embodiments, communications systems 518 can include any suitable hardware, firmware, and/or software for communicating information over communication network 454 and/or any other suitable communication networks. For example, communications systems 518 can include one or more transceivers, one or more communication chips and/or chip sets, and so on. In a more particular example, communications systems 518 can include hardware, firmware, and/or software that can be used to establish a Wi-Fi connection, a Bluetooth connection, a cellular connection, an Ethernet connection, and so on.

In some embodiments, memory 520 can include any suitable storage device or devices that can be used to store instructions, values, data, or the like, that can be used, for example, by processor 512 to present content using display 514, to communicate with one or more computing devices 450, and so on. Memory 520 can include any suitable volatile memory, non-volatile memory, storage, or any suitable combination thereof. For example, memory 520 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 520 can have encoded thereon a server program for controlling operation of server 452. In such embodiments, processor 512 can execute at least a portion of the server program to transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone), and so on.

In some embodiments, the server 452 is configured to perform the methods described in the present disclosure. For example, the processor 512 and memory 520 can be configured to perform the methods described herein (e.g., the method of FIG. 2).

In some embodiments, data source 402 can include a processor 522, one or more data acquisition systems 524, one or more communications systems 526, and/or memory 528. In some embodiments, processor 522 can be any suitable hardware processor or combination of processors, such as a CPU, a GPU, and so on. In some embodiments, the one or more data acquisition systems 524 are generally configured to acquire data, images, or both, and can include an ultrasound device (e.g., ultrasound device 102) and/or a CT imaging system. Additionally or alternatively, in some embodiments, the one or more data acquisition systems 524 can include any suitable hardware, firmware, and/or software for coupling to and/or controlling operations of the ultrasound device and/or CT imaging system. In some embodiments, one or more portions of the data acquisition system(s) 524 can be removable and/or replaceable.

Note that, although not shown, data source 402 can include any suitable inputs and/or outputs. For example, data source 402 can include input devices and/or sensors that can be used to receive user input, such as a keyboard, a mouse, a touchscreen, a microphone, a trackpad, a trackball, and so on. As another example, data source 402 can include any suitable display devices, such as an LCD screen, an LED display, an OLED display, an electrophoretic display, a computer monitor, a touchscreen, a television, etc., one or more speakers, and so on.

In some embodiments, communications systems 526 can include any suitable hardware, firmware, and/or software for communicating information to computing device 450 (and, in some embodiments, over communication network 454 and/or any other suitable communication networks). For example, communications systems 526 can include one or more transceivers, one or more communication chips and/or chip sets, and so on. In a more particular example, communications systems 526 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 528 can include any suitable storage device or devices that can be used to store instructions, values, data, or the like, that can be used, for example, by processor 522 to control the one or more data acquisition systems 524, and/or receive data from the one or more data acquisition systems 524; to generate images from data; present content (e.g., data, images, a user interface) using a display; communicate with one or more computing devices 450; and so on. Memory 528 can include any suitable volatile memory, non-volatile memory, storage, or any suitable combination thereof. For example, memory 528 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 528 can have encoded thereon, or otherwise stored therein, a program for controlling operation of data source 402. In such embodiments, processor 522 can execute at least a portion of the program to generate images, transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for analyzing ultrasound data to monitor agreement of an anatomical target morphology with a radiation treatment plan, the method comprising:
(a) acquiring ultrasound data from a region-of-interest in a patient containing an anatomical target, wherein the ultrasound data are acquired using at least one ultrasound transducer arranged about the region-of-interest;
(b) accessing with a computer system, reference ultrasound data acquired from the patient while the anatomical target was maintained at a specified reference morphology indicated by a radiation treatment plan;
(c) comparing the ultrasound data with the reference ultrasound data, generating feedback data that indicate a conformance of the anatomical target morphology to the reference morphology; and
(d) when the anatomical target morphology is optimally in agreement with the reference morphology, generating a notification and delivering the notification to the patient via a user device, wherein the notification indicates that the anatomical target morphology is optimally in agreement with the reference morphology indicated by the radiation treatment plan.

2. The method of claim 1, further comprising controlling a radiation treatment system to deliver radiation to the patient based on the feedback data indicating that the anatomical target morphology is optimally aligned with the reference morphology indicated by the radiation treatment plan.

3. The method of claim 1, wherein the specified reference morphology indicated in the reference ultrasound data is confirmed via at least one of computed tomography (CT) imaging of the patient or magnetic resonance imaging (MRI) of the patient.

4. The method of claim 1, wherein the ultrasound data are continually acquired from the patient and compared with the reference ultrasound data, thereby continually generating feedback data in real-time.

5. The method of claim 1, wherein the ultrasound data are intermittently acquired from the patient and compared with the reference ultrasound data, thereby continually generating feedback data in real-time according to an intermittent schedule.

6. The method of claim 1, wherein the anatomical target morphology is optimally in agreement with the reference morphology when a planned target volume (PTV) in the radiation treatment plan is in alignment with a radiation beam path while minimizing exposure of organs-at-risk (OARs) proximate to the anatomical target.

7. The method of claim 1, wherein the anatomical target is a bladder.

8. The method of claim 7, wherein the specified reference morphology indicates a bladder filling that results in optimally positioning a planned target volume (PTV) proximate the bladder in a radiation beam path while minimizing exposure to organs-at-risk (OARs) proximate the bladder.

9. The method of claim 8, wherein the specified reference morphology includes a size and shape of the bladder.

10. The method of claim 7, wherein the feedback data include a notification to the patient to drink more liquid to provide further filling of the bladder in order to conform the anatomical target morphology with the reference morphology.

11. The method of claim 1, wherein the anatomical target is a rectum.

12. The method of claim 11, wherein the specified reference morphology indicates a rectal emptying that results in optimally positioning a planned target volume (PTV) proximate the rectum in a radiation beam path while minimizing exposure to organs-at-risk (OARs) proximate the rectum.

13. The method of claim 11, wherein the feedback data include a notification to the patient to empty the rectum in order to conform the anatomical target morphology with the reference morphology.

14. The method of claim 1, wherein the anatomical target is a stomach.

15. The method of claim 14, wherein the specified reference morphology indicates a stomach emptying that results in optimally positioning a planned target volume (PTV) proximate the stomach in a radiation beam path while minimizing exposure to organs-at-risk (OARs) proximate the stomach.

16. The method of claim 14, wherein the feedback data include a notification to the patient to fast in order to conform the anatomical target morphology with the reference morphology.

17. The method of claim 1, wherein the feedback data generated by comparing the ultrasound data with the reference ultrasound data include a timer counting down a predicted time to when the anatomical target morphology will optimally align with the reference morphology.

18. The method of claim 1, further comprising receiving patient feedback data via the user device, wherein the patient feedback data indicate a comfort level of the patient based on a current morphology of the anatomical target.

19. The method of claim 1, wherein the ultrasound data are acquired from the region-of-interest in the patient using a plurality of ultrasound transducers arranged about the region-of-interest.

20. A user device, comprising:
a display;
a memory; and
a processor in communication with the display and the memory, the processor being configured to:
receive ultrasound data acquired from a region-of-interest in a patient containing an anatomical target, wherein the ultrasound data were acquired using a plurality of ultrasound transducer arranged about the region-of-interest;
access from the memory, reference ultrasound data acquired from the patient while the anatomical target was maintained at a specified reference morphology indicated by a radiation treatment plan;
compare the ultrasound data with the reference ultrasound data, generating feedback data that indicate a conformance of the anatomical target morphology to the reference morphology;
generate a user interface displayed to a user by the display; and
present the feedback data in the user interface displayed by the display.

21. The user device of claim 20, wherein the feedback data comprise a notification indicating when the anatomical target morphology is optimally aligned with the reference morphology indicated by the radiation treatment plan.

22. The user device of claim 20, wherein the feedback data comprise a timer indicating a predicted time until the anatomical target morphology will be optimally aligned with the reference morphology indicated by the radiation treatment plan.

23. The user device of claim 22, wherein the feedback data further comprise a notification to the user to one of drink more liquid, fast, or empty their rectum.

24. The user device of claim 20, wherein the processor is configured to receive patient feedback data via the user interface, wherein the patient feedback data indicate a comfort level of the patient based on a current morphology of the anatomical target.

25. The user device of claim 24, wherein the processor is configured to adjust an alignment target between the morphology of the anatomical target and the reference morphology based on the patient feedback data.

26. A method for monitoring agreement of an anatomical target morphology with a radiation treatment plan, the method comprising:
(a) accessing ultrasound data with a computer system, wherein the ultrasound data have been acquired from a region-of-interest in a patient containing an anatomical target;
(b) accessing reference ultrasound data with the computer system, wherein the reference ultrasound data have been acquired from the patient while the anatomical target was maintained at a specified reference morphology indicated by a radiation treatment plan;
(c) generating feedback data with the computer system by comparing the ultrasound data with the reference ultrasound data, wherein the feedback data indicate an agreement of the anatomical target morphology to the reference morphology; and
(d) outputting the feedback data to a user via the computer system.

27. The method of claim 26, wherein generating the feedback data further includes receiving patient feedback data from the patient via the computer system and updating the feedback data based on the patient feedback data.

28. The method of claim 26, wherein outputting the feedback data to the user comprises generating a notification with the computer system, wherein the notification indicates a measure of agreement between the anatomical target morphology and the reference morphology.
